(19) United States
(12) Patent Application Publication
(10) Pub. No.: US 2018/0088248 A1
(43) Pub. Date: Mar. 29, 2018

(54) X-RAY DETECTOR WITH THERMALLY-CONDUCTIVE INTERMEDIATE LAYER

(71) Applicant: Siemens Healthcare GmbH, Erlangen (DE)

(72) Inventors: Thorsten ERGLER, Erlangen (DE); Harald GEYER, Bubenreuth (DE); Jan WREGE, Erlangen (DE)

(73) Assignee: Siemens Healthcare GmbH, Erlangen (DE)

(21) Appl. No.: 15/697,511

(22) Filed: Sep. 7, 2017

(30) Foreign Application Priority Data
Sep. 23, 2016 (DE) 102016218338.9

(51) Int. Cl. G01T 1/24; H01L 27/146; A61B 6/03; A61B 6/00

(52) U.S. Cl. CPC G01T 1/244; H01L 27/14658; G01T 1/243; A61B 6/4488; A61B 6/4411; A61B 6/4208; A61B 6/032

(57) ABSTRACT

An x-ray detector includes, in a stack formation, a converter element, an evaluation unit and an intermediate layer. In an embodiment, the intermediate layer has a thermal conductivity of more than 0.5 W/mK, preferably more than 2 W/mK and more preferably more than 6 W/mK.

### PRIORITY STATEMENT

The present application hereby claims priority under 35 U.S.C. § 119 to German patent application number DE 102016218338.9 filed Sep. 23, 2016, the entire contents of which are hereby incorporated herein by reference.

### FIELD

At least one embodiment of the invention generally relates to an x-ray detector, a medical device and to a method for producing an x-ray detector, wherein the x-ray detector has a thermally-conductive intermediate layer.

### BACKGROUND

In x-ray imaging, for instance in computed tomography, angiography or radiography, counting directly converting x-ray detectors or integrated indirectly converting x-ray detectors can be used.

The x-ray radiation or the photons can be converted in directly converting x-ray detectors by a suitable converter material into electric pulses. CdTe, CZT, CdZnTeSe, CdTeSe, CdMnTe, InP, TlBr2, HgI2, GaAs or others can be used as the converter material, for example. The electric pulses are evaluated by an evaluation electronics system, for example an integrated circuit (Application Specific Integrated Circuit, ASIC). In counting x-ray detectors, incident x-ray radiation is measured by counting the electric pulses which are triggered by the absorption of x-ray photons in the converter material. The level of the electric pulse is generally proportional to the energy of the absorbed x-ray photon. By this means, spectral information can be extracted by comparing the level of the electric pulse with a threshold value.

The x-ray radiation or the photons can be converted in indirectly converting x-ray detectors by a suitable converter material into light and via photodiodes into electric pulses. Scintillators, for instance GOS (Gd2O2S), CsJ, YGO or LuTAG, are frequently used as converter material.

Scintillators are used particularly in medical x-ray imaging in the energy range up to 1 MeV. What are known as indirectly converting x-ray detectors, known as scintillator detectors, are typically used, in which the conversion of the x-ray or gamma radiation into electric signals takes place in two stages. In a first stage, the x-ray or gamma quanta are absorbed in a scintillator element and converted into optically visible light; this effect is referred to as luminescence. The light excited by luminescence is then converted in a second stage by a first photodiode, which is optically coupled to the scintillator element, into an electric signal, read out by way of an evaluation or read-out electronics system and then forwarded to the computing unit.

### SUMMARY

The inventors have recognized that a problem underlying at least one embodiment of the invention is that temperature changes in the x-ray detector negatively influence the measurement results. For instance, the resistance of the directly converting converter material can change with the x-ray flow; at the same time this may also result in a change in the current by the applied high voltage in the converter element and thus in a change in the power loss. This results in a temperature change which influences the count rate and energy resolution.

As such, these x-ray detectors may suffer from a temperature-dependent counting rate drift, which results in artifacts in the imaging. Since in the imaging with a computed tomography system the detected dose can change during a scan, this may be a time-dependent or dynamic effect. The inventors have recognized that the time-dependent effect can be compensated for by suitable temperature stabilization measures.

At least one embodiment of the invention specifies an x-ray detector, a medical device and/or a method for producing an x-ray detector, which permit an improved temperature stabilization of the converter element.

At least one embodiment of the invention is directed to an x-ray detector; at least one embodiment of the invention is directed to a medical device; and at least one embodiment of the invention is directed to a method for producing an x-ray detector.

At least one embodiment of the invention relates to an x-ray detector having a stack formation with a converter element, an evaluation unit and an intermediate layer, wherein the intermediate layer has a thermal conductivity of more than 0.5 W/mK, preferably more than 2 W/mK and particularly preferably more than 6 W/mK.

At least one embodiment of the invention further relates to a medical device including the embodiments of the inventive x-ray detector. Advantageously the advantages of the embodiments of the inventive x-ray detector can be transferred to the medical device.

At least one embodiment of the invention relates further to a method for producing an x-ray detector including: provisioning an evaluation unit and at least one of converter element and substrate; filling an intermediate space between the evaluation unit and at least the converter element or the substrate with a filler material; and hardening the filler material to generate an intermediate layer, wherein the intermediate layer has a thermal conductivity of more than 0.5 W/mK, preferably more than 2 W/mK, particularly preferably more than 6 W/mK. The intermediate space can be embodied between the converter element and the evaluation unit or between the evaluation unit and the substrate.

### BRIEF DESCRIPTION OF THE DRAWINGS

Example embodiments of the invention will now be described in more detail, making reference to the drawings, in which:

FIG. 1 shows a schematic representation of a concept of an inventive x-ray detector according to a first embodiment;

FIG. 2 shows a schematic representation of a concept of an inventive x-ray detector according to a second embodiment;

FIG. 3 shows a schematic representation of a concept of an inventive x-ray detector according to a third embodiment;

FIG. 4 shows a schematic representation of an inventive x-ray detector according to a fourth embodiment;

FIG. 5 shows a schematic representation of a concept of an embodiment of an inventive computed tomography system; and

FIG. 6 shows a schematic representation of a concept of an embodiment of an inventive method for producing an inventive x-ray detector.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to an x-ray detector having a stack formation with a converter element, an evaluation unit and an intermediate layer, wherein the intermediate layer has a thermal conductivity of more than 0.5 W/mK, preferably more than 2 W/mK and particularly preferably more than 6 W/mK.

In an embodiment, the converter element and the evaluation unit can be connected to one another in a stack formation. The evaluation unit may have an evaluation electronics system, for instance an integrated circuit. The preferably directly converting converter element and the evaluation unit can have at least one contact or one electrode, which can be electrically conductingly connected to one another via a soldered connection for instance. The converter element and the evaluation unit can preferably have a plurality of contacts, which, preferably on the converter element and the evaluation unit, have a similar distribution or a similar pattern with similar gaps for instance, so that the contacts of the converter element and the contacts of the evaluation unit lie congruently on top of one another in a stack formation. The converter element and the evaluation unit can have a similar number of contacts. The converter element and the evaluation unit can have a pixel-type structure.

The intermediate layer may be a slack-fill, known as an underfill. The intermediate layer can be filled in a flowable state following the electrically conducting connection of the converter element and the evaluation unit in an intermediate space embodied therebetween. The intermediate layer can solidify or harden after the filling process. The intermediate layer can be hardened in particular in a thermal process. The intermediate layer can have an intermediate layer material. The intermediate layer or the intermediate layer material can, for instance in the flowable state, have a viscosity between 3300 mPa·s and 65000 mPa·s. The intermediate layer material can be processed or filled in a flowable state particularly at a temperature in the range of 50° C. to 90° C.

The intermediate layer is arranged in a planar manner between the converter element and the evaluation unit. The converter element, the intermediate layer and the evaluation unit can form a stack formation. The planar arrangement of the intermediate layer may indicate that the intermediate layer extends substantially across the entire extent of the boundary with the evaluation unit and/or the boundary with the converter element. The intermediate layer may have an extent which differs from the converter element and/or from the evaluation unit. The intermediate layer may be at least partially transparent for visible, ultraviolet or infrared light.

The intermediate layer material may be adjusted to the thermal expansion coefficients of the converter element and/or the evaluation unit. The intermediate layer may have a thermal conductivity of more than 0.5 W/mK, more than 2 W/mK, more than 4 W/mK, more than 6 W/mK, more than 8 W/mK or more than 10 W/mK for instance. The intermediate layer material or the intermediate layer has a thermal conductivity of more than 0.5 W/mK, preferably more than 2 W/mK, particularly preferably more than 6 W/mK and in particular preferably more than 7 W/mK.

The higher the thermal conductivity of the intermediate layer, the more suitable the intermediate layer is for reaching a homogeneous temperature distribution in the converter element. The higher the thermal conductivity of the intermediate layer, the more suitable the intermediate layer is for heating or cooling the converter element. The thermal conductivity of the intermediate layer can be increased for instance by adding suitable fillers.

The intermediate layer can surround the electrically conducting connections, in particular soldered connections, between the converter element and the evaluation unit. The intermediate layer can have the electrically conducting connections. The intermediate layer can advantageously increase the mechanical stability of the stack formation. The intermediate layer can advantageously increase the stability with respect to temperature changes. The intermediate layer can protect against the penetration of moisture into an intermediate space.

The use of a particularly thermally-conductive intermediate layer material can ensure a more homogeneous temperature distribution and also an improved temperature flow on the converter element. An improved heat transmission can advantageously be permitted between the converter element and the evaluation unit. Advantageously the number of heating elements in the x-ray detector can be reduced. Advantageously costs can be saved when producing the x-ray detector.

At least one embodiment of the invention further relates to a medical device including the embodiments of the inventive x-ray detector. Advantageously the advantages of the embodiments of the inventive x-ray detector can be transferred to the medical device.

At least one embodiment of the invention relates further to a method for producing an x-ray detector including: provisioning an evaluation unit and at least one of converter element and substrate; filling an intermediate space between the evaluation unit and at least the converter element or the substrate with a filler material; and hardening the filler material to generate an intermediate layer, wherein the intermediate layer has a thermal conductivity of more than 0.5 W/mK, preferably more than 2 W/mK, particularly preferably more than 6 W/mK. The intermediate space can be embodied between the converter element and the evaluation unit or between the evaluation unit and the substrate.

In the provisioning step, a converter element and an evaluation unit are provided. The provisioning process can comprise for instance the converter element and the evaluation unit being connected via soldered connections. An intermediate space which can be filled can be embodied between the converter element and the evaluation unit. Furthermore or alternatively a substrate can be arranged on the side of the evaluation unit which faces away from the converter element, said substrate being connected via soldered connections to the evaluation unit. An intermediate space which can be filled can be embodied between the evaluation unit and the substrate.

In the step of filling, the intermediate space between the converter element and the evaluation unit and/or an intermediate space adjoining the side of the evaluation unit which faces away from the converter element is filled. In the step of filling, the filler material can be filled in a flowable state.

In the step of hardening, the filler material can be hardened for instance by way of thermal impact or from the impact of UV radiation. An intermediate layer is generated by the hardening process. The intermediate layer has a solidified filler material in the final state. The x-ray detector advantageously has an increased mechanical stability and an increased thermal conductivity.

According to one embodiment of the invention, the intermediate layer is arranged in a planar manner between the converter element and the evaluation unit and/or in a planar manner on the side of the evaluation unit which faces away from the converter element. The intermediate layer can be arranged in a planar manner between the converter element and the evaluation unit and possible units arranged therebetween.

The intermediate layer can further or alternatively be arranged on the side of the evaluation unit which faces away from the converter element. By way of example the intermediate layer can be arranged on a substrate on a side of the evaluation unit which faces away from the converter element, for instance between the substrate and the evaluation unit. The intermediate space can advantageously be filled and thus stabilized by the intermediate layer. Advantageously the thermal conduction between the converter element and evaluation unit or substrate and evaluation unit can be improved.

According to one embodiment of the invention, the x-ray detector also has a heating element. The heating element can be included in a unit of the x-ray detector. The heating element can be included in the evaluation unit for instance.

The heating element can be embodied for instance by the evaluation unit, for instance on a surface of the evaluation unit which faces away from the converter element. For instance, the heating element can be arranged in a rewiring layer of the evaluation unit. The heating element can be included in the substrate for instance. The heating element can be arranged on a side of the substrate which faces the evaluation unit for instance. The heating element can be arranged between the pad structures or electrodes for instance. The heating element can be arranged between the evaluation unit and the converter element for instance. The heating element can be arranged between the evaluation unit and substrate for instance. The heating element can be included in the converter element for instance. The heating element can be embodied on a side of the converter element which faces or faces away from the incident direction of the x-ray radiation during operation for instance.

A number of heating elements can be arranged in the x-ray detector. A number of heating elements can be operated in combination. An improved temperature distribution or a more homogeneous distribution of the heat toward the converter element can be achieved by way of the intermediate layer. As a result, a reduction in the previously required heating elements can be advantageously achieved. Advantageously the costs can be reduced. The closer the heating element is arranged to the converter element, the better a heat input or cooling effect can be achieved in the converter element.

The converter element can advantageously be kept at a temperature which is substantially constant by use of the heating element. Advantageously temperature fluctuations can be compensated for, which under the influence of x-ray radiation may comprise a higher temperature in the converter element and without the influence of x-ray radiation may comprise a cooling down of the converter element. Advantageously the converter element can be kept at the substantially constant temperature without or under the minimal influence of x-ray radiation. A count rate change can thus advantageously be reduced or prevented.

According to one embodiment of the invention, the intermediate layer has an epoxy compound, a plastic material, a composite material or a (pre) polymer. The intermediate layer may have a binding material. It may be a matrix embodied from binding material and filler. The intermediate layer or the intermediate layer material can have in particular an epoxy resin. At the time of filling the intermediate layer into the intermediate space, for instance between the converter element and evaluation unit, the material of the intermediate layer, for instance having an epoxy compound, an epoxy resin or a prepolymer, may be liquid or flowable. Advantageously the intermediate layer can harden under temperature effects for instance.

According to one embodiment of the invention, the intermediate layer has a filler with a thermal conductivity of more than 0.5 W/mK, preferably more than 2 W/mK, particularly preferably more than 6 W/mK. The filler can preferably be electrically insulated or non-conducting. The filler can have electrically conducting particles for instance, which are insulated such that the filler is electrically insulated. Advantageously no electrically conducting connection can be produced by arranging filler particles in series.

The intermediate layer material can have a filler. The filler can have a low, in particular thermal, expansion coefficient. The intermediate layer can in addition to the filler have further fillers such as for instance Al₂O₃, SiO₂, BN, AlN, TiN, TiO₂, PZT (PbZrTiO₃), ZrO₂ or YSZ (known as Yttria-stabilized zirconia). The filler can advantageously contribute to the mechanical stability of the stack formation.

The concentration of the filler can be chosen such that the viscosity of the intermediate layer material, for instance in the flowable state, amounts to between 3300 mPa·s and 65000 mPa·s. The diameter or the size of the filler particles of the filler may in particular be smaller than the distance between the converter element or the substrate and the evaluation unit, for instance smaller than 33 percent, preferably 20 percent and particularly preferably 10 percent of the distance.

In order to adjust the thermal expansion coefficient, the filler can be advantageously adjusted to the adjacent units, for instance the converter element, the evaluation unit or the substrate. The shape of the filler particles can be spherical, round, angular or flakey for instance. The targeted use of fillers with a high thermal conductivity such as for instance diamond, nanoparticles, graphene or carbon nanotubes can advantageously increase the thermal conductivity of the intermediate layer. The thermal expansion coefficient of the intermediate layer or of the binding material can be less than 100 ppm/K and in particular less than 50 ppm/K and preferably in the range of 25 to 30 ppm/K for instance.

According to one embodiment of the invention, the filler is distributed uniformly in the intermediate layer. Advantageously a homogeneous and improved heat distribution in the x-ray detector can be permitted. Advantageously the temperature of the converter element can be stabilized in a simplified manner.

According to one embodiment of the invention, the filler has diamond. The intermediate layer with diamond as a filler can have a thermal conductivity of 6 W/mK for instance. The thermal conductivity of the intermediate layer can advantageously be increased by more than the factor 10.

According to one embodiment of the invention, the filler has nanoparticles. The filler can have filler particles. The filler can have filler particles with an extent in the nanometer range. The filler can have nanotubes, fullerenes or flakes for instance. The maximum extent, for instance the length, of the filler particles should amount at most to 33 percent of the distance between the evaluation unit and the converter element or the substrate, for instance a maximum of 20 μm. The filler can have carbon nanotubes for instance.

According to one embodiment of the invention the filler has superficially insulated metal particles. Metal particles can advantageously be used as a filler in order to increase the thermal conductivity. In such cases the surface of the metal particles can be insulated by a superficial polymer coating for instance.

According to one embodiment of the invention, the filler has graphene. The graphene may be present as a filler in a powdered or flake-type state for instance. Advantageously the thermal conductivity of the intermediate layer can be increased.

According to one embodiment of the invention, the converter element has a directly converting converter material. According to one embodiment of the invention, the converter element has cadmium. The converter element may have CdZnTe, CdTe or CdHgTe. Advantageously the converter element may be suitable for converting x-ray radiation in the field of medical x-ray imaging. The use of a thermally-conductive intermediate layer is particularly advantageous in directly converting x-ray detectors.

According to one embodiment of the invention, the medical device is a computed tomography system. Advantageously slice images, three-dimensional or four-dimensional volume images can be reconstructed with the aid of the measurement data of the x-ray detector.

The FIG. 1 shows an example embodiment of the inventive x-ray detector 1 according to a first embodiment. The x-ray detector 1 has for instance a directly converting converter element 3, an evaluation unit 15 and an intermediate layer 11 arranged in a planar manner between the directly converting converter element 3 and the evaluation unit 15, wherein the intermediate layer 11 has a thermal conductivity of more than 0.5 W/mK. The x-ray radiation 21 strikes the converter element 3 during operation. A contact on the converter element 3 and a contact on the evaluation unit 15 are connected to at least one soldered connection 9 and the at least one soldered connection 9 is partially surrounded by the intermediate layer 11. The intermediate layer 11 is arranged in a planar manner between the converter element 3 and the evaluation unit 15. The intermediate layer 11 has an epoxy compound, a plastic material or a polymer. The intermediate layer 11 has a filler with a thermal conductivity of more than 0.5 W/mK, preferably of more than 6 W/mK. The filler is distributed uniformly in the intermediate layer 11. The filler has diamond, nanoparticles, superficially insulated metal particles or graphene.

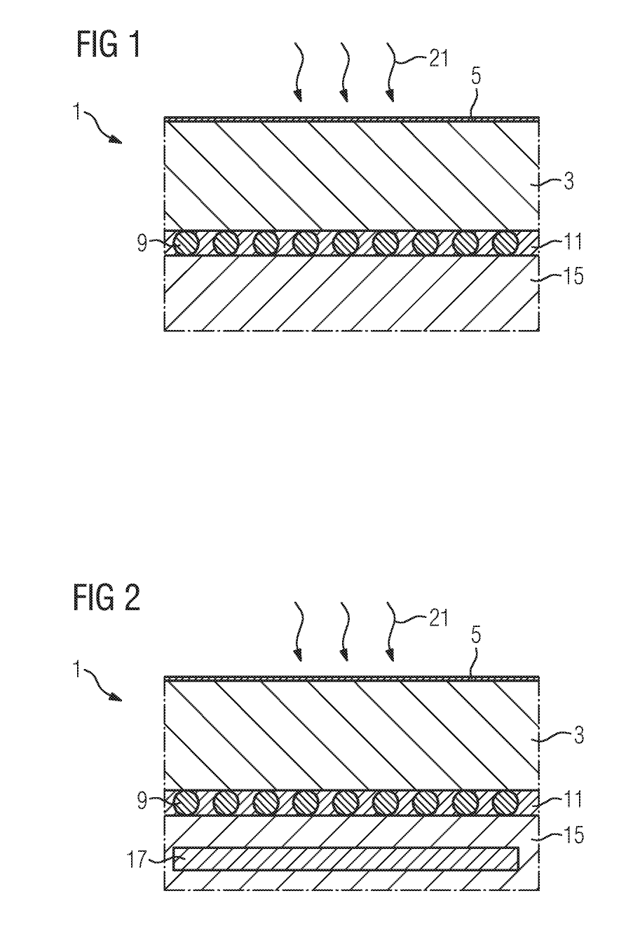

FIG. 2 shows an example embodiment of the inventive x-ray detector 1 according to a second embodiment. The x-ray detector 1 further has a heating element 17. The heating element 17 is included in the evaluation unit 15. The heating element 17 can be embodied for instance on a surface of the evaluation unit 15 which faces away from the converter element 3. For instance, the heating element 17 can be arranged in a rewiring layer of the evaluation unit 15.

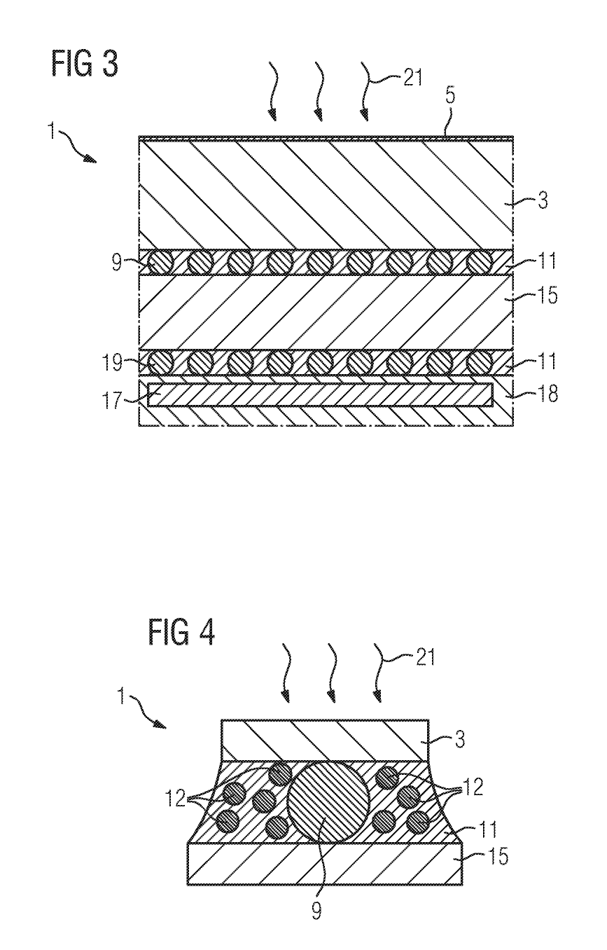

FIG. 3 shows an example embodiment of the inventive x-ray detector 1 according to a third embodiment. The intermediate layer 11 is arranged in a planar manner between the converter element 3 and the evaluation unit 15. The intermediate layer 11 is further arranged in a planar manner between the side of the evaluation unit 15 and the substrate 18 which faces away from the converter element 3. Further soldered connections 19 connect the evaluation unit 15 and the substrate 18 in an electrically conducting manner. The heating element 17 can be included in the substrate 18 for instance. The heating element 17 can be arranged on a side of the substrate 18 which faces the evaluation unit 15 for instance.

FIG. 4 shows an example embodiment of the inventive x-ray detector 1 according to a fourth embodiment. The intermediate layer 11 has a filler 12. The filler 12 has a minimal, especially thermal expansion coefficient. The intermediate layer 11 has a filler 12 with a thermal conductivity of more than 0.5 W/mK, preferably of more than 6 W/mK. The concentration of the filler 12 is chosen such that the viscosity of the intermediate layer material, for instance in the flowable state, amounts to a viscosity between 3300 mPa·s and 65000 mPa·s. The intermediate layer material can be processed or filled in a flowable state particularly at a temperature in the range of 50° C. to 90° C. The diameter or the size of the filler particles of the filler 12 is in particular smaller than the distance between the converter element 3 and the evaluation unit 15, for instance smaller than 33 percent of the distance. The intermediate layer can in addition to the filler 12 have further fillers such as for instance Al₂O₃, SiO₂, BN, AlN, TiN, TiO₂, PZT (PbZrTiO₃), ZrO₂ or YSZ (known as Yttria-stabilized zirconia). The planar extent of the converter element 3 and the planar extent of the evaluation unit 15 may differ. The intermediate layer 11 can be laterally delimited by concave surfaces.

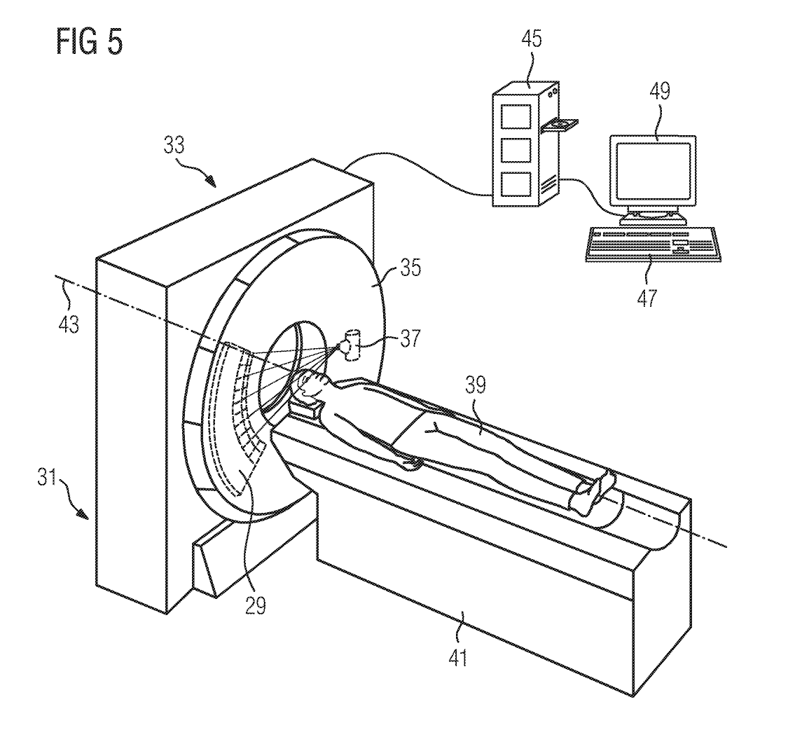

FIG. 5 shows an example embodiment of the inventive computed tomography system 31 with a detector apparatus 29. The detector apparatus 29 has the inventive x-ray detector. The computed tomography system 31 contains a gantry 33 with a rotor 35. The rotor 35 comprises an x-ray source 37 and the inventive detector apparatus 29. The patient 39 is supported on the patient couch 41 and can be moved along the axis of rotation z 43 through the gantry 33. A computing unit 45 is used to control and calculate the cross-sectional images. An input facility 47 and an output apparatus 49 are connected to the computing unit 45.

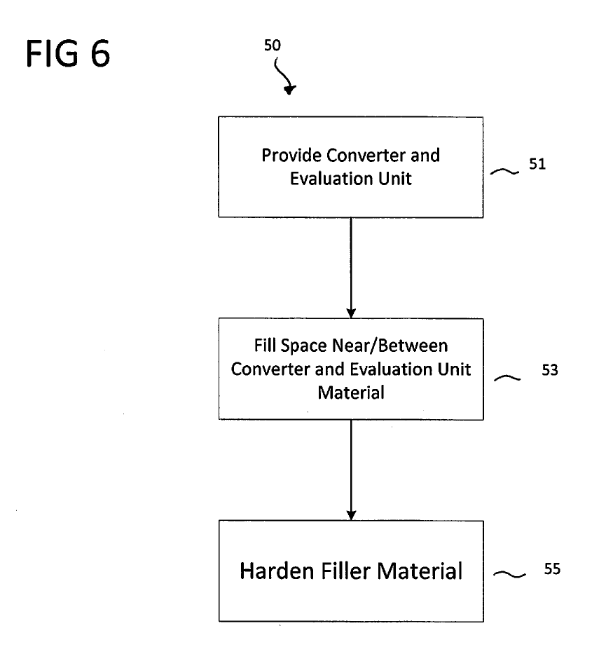

FIG. 6 shows an example embodiment of the inventive method 50 for producing an inventive x-ray detector. In the provisioning step 51, a converter element and an evaluation unit are provided. The provisioning 51 can comprise for instance the converter element and the evaluation unit being connected via soldered connections. An intermediate space which can be filled can be embodied between the converter element and the evaluation unit. Furthermore or alternatively a substrate can be arranged on the side of the evaluation unit which faces away from the converter element, said substrate being connected via soldered connections to the evaluation unit for instance. An intermediate space which can be filled can be embodied between the evaluation unit and the substrate. In the filling step 53 the intermediate space between the converter element and the evaluation unit and/or an intermediate space adjacent to the side of the evaluation unit which faces away from the converter element are filled. In the filling step 53 the filler material can be filled in a flowable state. The concentration of the filler can be chosen such that the viscosity of the intermediate layer material, for instance in the flowable state, amounts to between 3300 mPa·s and 65000 mPa·s for instance. In the hardening step 55, the filler material can be hardened for instance by way of thermal impact or under the impact of UV radiation. An intermediate layer is generated by the hardening process 55. The intermediate layer has a solidified filler material in the final state. The intermediate layer 11 has a thermal conductivity of more than 0.5 W/mK in the final state.

Although the invention has been disclosed in detail with the preferred example embodiment, the invention is not restricted by the examples given and other variations can be derived therefrom by a person skilled in the art without departing from the protective scope of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An x-ray detector comprising, in a stack formation, a converter element, an evaluation unit and an intermediate layer, the intermediate layer having a thermal conductivity of more than 0.5 W/mK.

2. The x-ray detector of claim 1, wherein the intermediate layer is arranged at least one of
in planar manner between the converter element and the evaluation unit and
in a planar manner on a side of the evaluation unit facing away from the converter element.

3. The x-ray detector of claim 1, further comprising a heating element.

4. The x-ray detector of claim 1, wherein the intermediate layer includes an epoxy compound or a polymer.

5. The x-ray detector of claim 1, wherein the intermediate layer includes a filler with a thermal conductivity of more than 0.5 W/mK.

6. The x-ray detector of claim 5, wherein the filler is distributed uniformly in the intermediate layer.

7. The x-ray detector of claim 5, wherein the filler includes diamond.

8. The x-ray detector of claim 5, wherein the filler includes nanoparticles.

9. The x-ray detector of claim 5, wherein the filler includes superficially insulated metal particles.

10. The x-ray detector of claim 5, wherein the filler includes graphene.

11. The x-ray detector of claim 1, wherein the converter element includes a directly converting converter material.

12. The x-ray detector of claim 11, wherein the converter element includes cadmium.

13. A medical device comprising the x-ray detector of claim 1.

14. The medical device of claim 13, wherein the medical device is a computed tomography system.

15. A method comprising:
providing an evaluation unit, and providing at least one of a converter element and a substrate;
filling an intermediate space, between the evaluation unit and the at least of the converter element and the substrate, with a filler material; and
hardening the filler material to generate an intermediate layer, the intermediate layer including a thermal conductivity of more than 0.5 W/mK.

16. The x-ray detector of claim 2, further comprising a heating element.

17. The x-ray detector of claim 6, wherein the filler includes diamond.

18. The x-ray detector of claim 5, wherein the converter element includes a directly converting converter material.

19. The x-ray detector of claim 18, wherein the converter element includes cadmium.

20. A medical device comprising the x-ray detector of claim 3.

21. The medical device of claim 20, wherein the medical device is a computed tomography system.

22. A medical device comprising the x-ray detector of claim 5.

23. The medical device of claim 22, wherein the medical device is a computed tomography system.

24. The method of claim 15, further comprising
producing an x-ray detector from the evaluation unit, intermediate layer and the at least one of the converter element and the substrate.

25. The method of claim 15, wherein the providing of the at least one of a converter element and a substrate includes providing a converter element and a substrate and wherein the method further comprises
producing an x-ray detector from the evaluation unit, intermediate layer, the converter element and the substrate.

26. The x-ray detector of claim 1, wherein the intermediate layer includes a thermal conductivity of more than 2 W/mK.

27. The x-ray detector of claim 26, wherein the intermediate layer includes a thermal conductivity of more than 6 W/mK.

28. The method of claim 15, wherein the intermediate layer includes a thermal conductivity of more than 2 W/mK.

29. The method of claim 28, wherein the intermediate layer includes a thermal conductivity of more than 6 W/mK.
